SEQUENCE LISTING (6 sequences)

SEQ ID NO: 1 - DNA, 357 bp, Adenovirus
tagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagctgacg

SEQ ID NO: 2 - DNA, 198 bp, herpes simplex virus 1
tcccgcggccccgccccccacgcccgccgccgcgcgcgcgcacgccgcccggaccgccgcccgccttttttgcgcgcgcgcgcgcccgcggggggcccgggctgcccgccgccgccgctttaaagggccgcgcgcgacccccggggggtgtgttttggggggggcccgttttcggggtctggccgctcctccccccgg

SEQ ID NO: 3 - protein, 594 aa, Trichoplusia ni
MGSSLDDEHILSALLQSDDELVGEDSDSEISDHVSEDDVQSDTEEAFIDEVHEVQPTSSGSEILDEQNVIEQPGSSLASNKILTLPQRTIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVTKKRTYCTYCPSKIRRKANASCKKCKKVICREHNIDMCQSCF

SEQ ID NO: 4 - DNA, 21 bp, Artificial Sequence
  PCR primer
tggctgttaaaggcagtttgg

SEQ ID NO: 5 - DNA, 23 bp, Artificial Sequence
  PCR primer
gcactccattttcgtcaaatctt

SEQ ID NO: 6 - DNA, 33 bp, Artificial Sequence
  Taqman probe
tccaatatctggaacagttcaaagtgctcatct

(19) United States
(12) Patent Application Publication
(10) Pub. No.: US 2020/0157567 A1
(43) Pub. Date: May 21, 2020

(54) Viral Vector

(71) Applicant: Oxford Genetics Limited, Oxford Oxfordshire (GB)

(72) Inventors: Ryan Cawood, Oxford Oxfordshire (GB); Weiheng Su, Oxford Oxfordshire (GB)

(21) Appl. No.: 16/604,362

(22) PCT Filed: Apr. 11, 2018
(86) PCT No.: PCT/GB2018/050961

§ 371 Date: Oct. 10, 2019

(30) Foreign Application Priority Data
Apr. 12, 2017 (GB) 1705927.0

(51) Int. Cl. C12N 15/86; C12N 7/00

(52) U.S. Cl. CPC C12N 15/86; C12N 2710/10343; C12N 7/00

(57) ABSTRACT

The present invention relates to a viral vector comprising a transposon and a nucleic acid encoding a transposase. The transposon comprises a transgene, or insertion site for a transgene, for integration into the genome of a target cell. The expression of the transposase is controlled such that the transposase is not expressed during production or packaging of the viral vector. Furthermore, the transposon comprises a packaging signal for the virus genome, thus preventing the packaging of any viral genome from which the transposon has been removed. Also provided is a process for producing a modified mammalian cell and a process for producing a mammalian cell with a modified genome, using a viral vector of the invention.

### CROSS-REFERENCE

This application is a 371 U.S. national phase of PCT/GB2018/050961, filed Apr. 11, 2018, which claims priority from GB patent application no. 1705927.0, filed Apr. 12, 2017, both which are incorporated by reference in its entirety.

### FIELD OF THE INVENTION

The present invention relates to a viral vector comprising a transposon and a nucleic acid encoding a transposase. The transposon comprises a transgene, or insertion site for a transgene, for integration into the genome of a target cell. The expression of the transposase is controlled such that the transposase is not expressed during production or packaging of the viral vector. Furthermore, the transposon comprises a packaging signal for the virus genome, thus preventing the packaging of any viral genome from which the transposon has been removed. Also provided is a process for producing a modified mammalian cell and a process for producing a mammalian cell with a modified genome, using a viral vector of the invention.

### BACKGROUND OF THE INVENTION

The delivery of genes to cells using viral vectors is an increasingly popular approach for many therapies for both genetic and infectious disease. However, the gene delivery vehicles used in these approaches must fulfil a number of criteria in order to be viable platform technologies. These include the ability to package significant quantities (e.g. 5 Kb) of exogenous DNA to be delivered; a high production yield to make a viable product that can be manufactured using a simple process; good stability at a range of temperatures; and being amenable to genetic engineering. In most cases, it is also desirable that the gene delivery vehicle is capable of integrating the DNA into infected cells.

Adenovirus fulfils many of the properties of an ideal gene delivery vehicle: it can be manufactured at high-yield; the genetics are well-characterised and understood; and the viral particles are more stable than many other (particularly enveloped) virus types. However, adenovirus does not integrate its DNA into the host chromosome to allow long-term stable expression. Other viral vectors do have this property, such as lentiviral and retroviral particles. These latter vectors are particularly useful for applications where pre-cursor cells are genetically modified using the vectors, wherein those cells will then expand in vivo. This is because if the DNA is not integrated, it can be lost during cell division from vectors that are maintained as episomes.

Previous attempts have been made to use the high-efficiency of adenoviral DNA delivery to cells with transposon-based systems (e.g. Yant et al., 2002, Nature Biotechnology, 20: 999-1005; Cooney et al., 2015, Molecular Therapy, 24(4): 667-674; Smith et al., 2015, Human Gene Therapy, 26: 377-385; and Chen et al., 2015, Genes Dis. 2(1): 96-105).

DNA transposons are natural and easily-controllable DNA delivery vehicles that can be used as tools for versatile gene delivery and gene discovery applications ranging from transgenesis to functional genomics and gene therapy. Transposons are simply organized: they encode a transposase protein in their simple genome flanked by inverted terminal repeats (ITRs) that carry transposase binding sites necessary for transposition. Transposons move through a “cut-and-paste” mechanism that involves excision of the element from the DNA and subsequent integration of the element into a new sequence environment.

One example of such a transposon is the piggyBac transposon. This transposon was originally isolated from the cabbage looper moth, Trichoplusia ni; it has been recognized as one of the most efficient DNA transposons for manipulating mammalian genes. The piggyBac transposon system has two major components: a donor plasmid (or transfer vector), carrying the gene of interest flanked by two terminal repeat domains; and a helper plasmid expressing the piggyBac transposase which catalyses the movement of the transposon. The piggyBac transposon has several distinct advantages over the lentiviral and/or retroviral systems, such as large cargo size, multiple copy integration, and it leaves no footprint.

However, these previous transposon-based systems have the disadvantage that they require two delivery vehicles. This means that the cost of developing these technologies is high because two agents are required, and the two agents must be combined to form a therapeutic product that is consistent between patient doses; this is more challenging when both genetic components are interdependent. For example, one cell may receive the DNA for the transposase only, or the ITRs containing DNA only, thereby reducing the activity of the system, and creating a heterogenous final system in terms of its functionality.

A desirable system would allow both the transposase and the ITRs to be encoded within the same DNA molecule for simultaneous delivery to cells. However, if the transposase is encoded within the same vector as the sequence to be transposed, then during production or manufacture, the transposase will transpose the sequence that is intended for final delivery to cells. A vector is therefore produced which does not contain any DNA for delivery to the target cells.

### SUMMARY OF THE INVENTION

In the invention described herein, undesired transposase activity is prevented by incorporating a packaging signal for the virus genome between the ITR sequences. Thereby, if during production the transposase is able to remove the ITR-flanked DNA from the virus genome, it cannot be packaged because the transposed sequence contains the sequences which are essential for virus packaging. Therefore, only full-length viral genomes can be packaged that contain the DNA to be transposed. Furthermore, the expression of the transposase is inducible or repressed, thus ensuring that transpose expression is only provided when desired.

It is therefore an object of the invention to provide a viral vector wherein a transposon comprising a transgene and a transposase are provided within a single vector, thus benefiting from the economies of a single-vector system and ensuring that each recipient cell receives both transposon and transposase.

It is another object of the invention to provide a process for producing a modified mammalian cell and a process for producing a mammalian cell with a modified genome, using the single-vector system of the invention.

### DESCRIPTION OF THE DRAWINGS

FIG. 1 shows a circular DNA map of an embodiment of the invention. The features in the DNA are highlighted including the relative positions of the virus and transposon ITRs and the virus packaging signal. The coding region for the transposase is shown to be positioned outside of the region that is transposed by the transposase.

FIG. 2 shows a linear DNA map of an embodiment of the invention with DNA features labelled to show their relative positions.

### DETAILED DESCRIPTION OF THE INVENTION

In one embodiment, the invention provides a viral vector comprising:
(a) a transposon comprising inverted terminal repeats (ITRs) at its 5′- and 3′-ends, the ITRs flanking
   (i) a packaging signal for the viral vector genome, and
   (ii) a transgene or a site for insertion of a transgene;
   and
(b) a nucleic acid encoding a transposase, wherein the transposase is one which is capable of recognising the ITRs of the transposon,
   and
   wherein the nucleic acid encoding the transposase is not flanked by the transposon ITRs.

Preferably, the nucleic acid encoding the transposase is operably associated with one or more transcriptional and/or translational control elements, more preferably with an inducible or suppressible promoter element.

In a further embodiment, the invention provides a process for producing a modified mammalian cell, the process comprising the step:
(a) infecting a mammalian cell with a viral vector of the invention, whereby the mammalian cell then comprises the viral vector.

In yet a further embodiment, the invention provides a process for producing a mammalian cell with a modified genome, the process comprising the steps:
(a) infecting a mammalian cell with a viral vector of the invention, wherein the transposon comprises a transgene; and
(b) inducing expression of the transposase

or removing repression of the transposase in the mammalian cell,

whereby the transposase excises the transposon from the viral vector and integrates the transposon into the genome of the mammalian cell.

The viral vector of the invention comprises a transposon and a nucleic acid encoding a transposase.

The viral vector may be used to introduce a desired transgene into the genome of a target cell (in which the viral vector is present). For example, upon infection of the viral vector into a mammalian cell and activation of the transposase, the transposon may integrate into the genome of that cell.

As used herein, the term “genome of a target cell” refers preferably to one or more of the chromosomes of the target mammalian cell. The genome may also be the mitochondrial genome.

The viral vector is preferably based on or derived from the genome of a double-stranded DNA virus. Preferably, the viral vector is replication-defective or replication-incompetent. Examples of such DNA viruses include those of the family Adenoviridae.

The adenovirus genome is a linear, 36 Kb double-stranded DNA (dsDNA) molecule containing multiple, heavily-spliced transcripts. At either end of the genome are inverted terminal repeats (ITRs). Its genes are divided into early (E1-4) and late (L1-5) transcripts. There are 57 accepted human adenovirus types.

Preferably, the viral vector is an adenoviral vector. Preferably, the adenoviral vector has the early genes E1 and E3 deleted. E1 may then be supplied by the adenovirus packaging lines 293, PerC6 or 911; its deletion from the viral vector renders the virus replication incompetent. E3 is involved in evading host immunity and is not essential for virus production. Deletion of these two components results in a transgene packaging capacity of >8 Kb. In some embodiments, the E2b gene is also deleted.

Preferably, the adenoviral vector is based on adenovirus serotype 5 (Ad5). Ad5-based vectors use the Coxsackie-Adenovirus Receptor (CAR) and the Alpha and Beta integrins to enter cells. Ad5-based vectors comprise, inter alia, Ad5 ITRs (which are distinct from the transposon ITRs) and the Ad5 virus packaging signal (which, in the context of this invention, is flanked by the transposon's ITRs).

Adenovirus Ad5 vectors are well known in the art. They have been modified to allow infection into new cell types where CAR is absent, such as T-cells. Such modifications have included the additional of a bi-specific antibody that binds to the surface of the virus and also binds to receptors on the surface of T-cells (e.g. CD3), thereby allowing the virus to infect T-cells (Wickham et al. Virol. 1997 October; 71(10): 7663-7669).

The virus may also be modified genetically by exchanging the fibre protein with that from another adenovirus serotype (such as AD35) to infect via the surface receptor CD46 (Yu et al., Anticancer Res. 2007 July-August; 27(4B):2311-6) or by modifying the fibre to contain RGD motifs.

Adenovirus may also be targeted to new cell types by the addition of new amino acid sequences with new binding specificities into the hypervariable region of the adenovirus Hexon protein. An example of this is the additional of a region of the VSVG glycoprotein coding sequence into the hexon hypervariable loops to provide broad virus tropism (Cho et al., Molecular Therapy, Vol. 7, Issue 5, Supplement, pS467, May 2003).

In other embodiments, the viral vector is a herpes virus vector. Herpes simplex virus (HSV) encodes more than 80 viral gene products and about half of these are essential for viral replication (Lachmann, “Herpes simplex virus-based vectors”, Int. J. Exp. Pathol. 2004 August; 85(4): 177-190). A replication-defective mutant may be produced by deleting one of the essential genes and providing the essential gene product in trans by means of a complementing cell line. Such mutant viruses are unable to replicate in non-complementing cells, but they remain cytotoxic due to the fact that they still express many viral gene products. Even mutant viruses deleted for the essential IE3 (ICP4) gene, which cannot initiate E and L gene expression, are cytotoxic. This toxicity is due to the expression of the other IE gene products. Therefore, the best way to minimize vector toxicity is to prevent viral gene expression altogether. Three different strategies have been used to generate such vectors.

First, herpes viral vectors may be constructed based on conditional mutations in a number of viral gene products. For example, a mutation in the virion transactivator, VP16, has been produced which prevents it from forming a protein-DNA complex and trans-inducing IE gene expression. This virus has a very high particle to plaque-forming unit (pfu) ratio and is avirulent when injected into mice. At high multiplicity of infection, however, it is still capable of undergoing full productive replication. Further mutations may be introduced, such as a temperature-sensitive mutation in ICP4 (tsK) and disabling mutations in ICP0. The resulting vectors can be propagated to high titres in permissive conditions using BHK cells, which have an endogenous activity which complements the ICP0defect, at 31° C., the permissive temperature for the tsK mutation, with the addition of hydroxymethyl bisacetamide, a transcriptional activator which complements the VP16 defect. In non-permissive conditions, however, infection at a multiplicity of 5 pfu per cell resulted in no detectable cytopathology and the persistence of the viral genome within cells.

The second approach has been to delete all of the IE genes from the viral genome and to construct a complementing cell line to provide the gene products which are necessary for efficient viral growth (ICP0 and the two essential IE products ICP4 and ICP27). The virus d109, which has been deleted for all five IE genes, could be used to infect non-complementing cells at MOIs of up to 30 with no detectable cytotoxicity.

The third strategy is to use HSV based amplicons where DNA is packaged into a viral particle by the nature of it containing the packaging signal of the HSV virus. DNA containing this sequence is amplified via a rolling circle mechanism and can be achieved by providing the HSV virus proteins in trans that allow the DNA to be packaged into infectious gene delivery particles.

Preferably, therefore, the herpes viral vector is fully replication-disabled. More preferably, it additionally lacks cytotoxicity.

The transposon is preferably a Class II DNA transposon. These generally move by a cut-and-paste mechanism in which the transposon is excised from one location and reintegrated elsewhere. The transposon is a non-autonomous transposon.

Complete DNA transposons generally consist of a transposase gene that is flanked by two Inverted Terminal Repeats (ITRs). The transposase recognizes these ITRs to perform the excision of the transposon DNA body, which is inserted into a new genomic location. Upon insertion, some target site DNA is duplicated, resulting in target site duplications, which represent a unique hallmark for each DNA transposon. In the context of this invention, however, the nucleic acid encoding the transposase is not present within the transposon.

As used herein in the context of the viral vector of the invention, the term “transposon” refers to the element (a) which is bounded by the transposon ITRs and which comprises the viral packaging signal and either a transgene or a site for insertion of a transgene.

Preferably, the transposon is one which is capable of integrating into a vertebrate genome, more preferably a mammalian genome. In other words, the transposon (and transposase) is preferably one which is active in vertebrate cells, more preferably in mammalian cells.

DNA transposons are classified into different families depending on their sequence, Inverted Terminal Repeats, and/or target site duplications. The families in Subclass I are: Tc1/mariner, PIF/Harbinger, hAT, Mutator, Merlin, Transib, P, piggyBac and CACTA, and Sleeping Beauty.

Helitron and Maverick transposons belong to Subclass II, since they are replicated and do not perform double-strand DNA breaks during their insertion. (See Muñoz-López and García-Pérez, “DNA Transposons: Nature and Applications in Genomics”, Current Genomics. 2010 April; 11(2): 115-128, for a review of DNA transposons).

Preferably, the transposon is based upon a Class II, Subclass I transposon.

Preferably, the transposon is based upon a piggyBac, Sleeping Beauty or a Tol2 transposon, or a variant or derivative thereof.

A review of the merits of the piggyBac, Sleeping Beauty and Tol2 transposon systems may be found in Grabundzjia et al., (2010) “Comparative analysis of transposable element vector systems in human cells”, Molecular Therapy, vol. 18, no. 6: 1200-1209.

In one embodiment, the transposon is based upon a piggyBac transposon, or a derivative thereof, and the transposase is a piggyBac transposase, or a derivative thereof.

piggyBac is a DNA transposon which was initially identified in the genome of the Cabbage Looper moth, Trichoplusia ni (Fraser, M J, et al., (1985), “Transposon-mediated mutagenesis of a baculovirus”, Virology 145: 356-361).

The original piggyBac transposon is 2.4 kb in length, contains 13 bp terminal inverted repeats, and additional 19 bp internal inverted repeats located asymmetrically with respect to the ends. Its target insertion site is TTAA.

Whilst the original piggyBac transposon comprises a single ORF (1.8 kb) that encodes a functional transposase, in the context of this invention, the nucleic acid encoding the piggyBac transposase is not present in the piggyBac transposon.

In the context of the present invention, the term “piggyBac transposon” refers to a transposon which comprises 5′ and 3′ terminal repeats from a piggyBac transposon.

In some embodiments, the gene which encodes the piggyBac transposase is the mouse codon-optimized PB transposase gene, mPB (Cadiianos and Bradley, (2007), “Generation of an inducible and optimized piggyBac transposon system”, Nucleic Acids Res 35: e87).

In other embodiments, the piggyBac transposase is native insect transposase, iPB (Li et al., PNAS, 2013, v110(25): E2279-E2287).

In one embodiment, the transposon is based upon a Sleeping Beauty transposon and the transposase is a Sleeping Beauty transposase, SB (Ivics, Z., et al., (1997), Molecular reconstruction of Sleeping Beauty, a Tc1-like transposon from fish, and its transposition in human cells”, Cell 91: 501-510).

In the context of the present invention, the term “Sleeping Beauty transposon” refers to a transposon which comprises 5′ and 3′ terminal repeats from a Sleeping Beauty transposon.

In the context of this invention, the nucleic acid encoding the Sleeping Beauty transposase is not present within the Sleeping Beauty transposon.

In some embodiments, the Sleeping Beauty transposase is SB or SB10, or a variant thereof having transposase activity (Ivics et al., supra).

In some embodiments, the Sleeping Beauty transposase is the hyperactive SB transposase, SB100X (Mátés, L., et al., (2009), “Molecular evolution of a novel hyperactive Sleeping Beauty transposase enables robust stable gene transfer in vertebrates”, Nat. Genet. 41: 753-761).

In one embodiment, the transposon is Tol2. The Tol2 transposon is a member of the hAT superfamily. It is endogenous in the medaka fish (Orizyas latipes; Koga, A., et al., (1996), “Transposable element in fish”, Nature 383: 30). Tol2 is the preferred transposon system for zebrafish. In the context of the present invention, the term “Tol2 transposon” refers to a transposon which comprises 5′ and 3′ terminal repeats from a Tol2 transposon.

As used herein the terms “inverted terminal repeats” and “terminal inverted repeats” are used interchangeably. The ITRs are transposon ITRs, i.e. they are derived from ITR sequences from a transposon. The ITRs are recognised by the transposase enzyme from the transposon from which the ITR is derived. Preferably, the transposon ITRs and the transposase are both obtained or derived from the same (wild-type) transposon.

ITRs contain a series of sub-sequences that are essential for transposon activity. In the case of the Sleeping Beauty transposon, this includes two imperfect direct repeats (DRs) of about 32 bp (Cui et al., Journal of Molecular Biology, volume 318, Issue 5, 17 May 2002, pp. 1221-1235). The outer DRs are at the extreme ends of the transposon, whereas the inner DRs are located inside the transposon, 165-166 bp from the outer DRs. Although there is a core transposase-binding sequence common to all of the DRs, additional adjacent sequences are required for transposition and these sequences vary in the different DRs. As a result, Sleeping Beauty transposase binds less tightly to the outer DRs than to the inner DRs. Two DRs are required in each ITR for transposition, but they are not interchangeable for efficient transposition. Each DR appears to have a distinctive role in transposition. The spacing and sequence between the DR elements in an ITR affect transposition rates, suggesting a constrained geometry is involved in the interactions of SB transposase molecules in order to achieve precise mobilization.

Transposons are flanked by TA dinucleotide base-pairs that are important for excision; elimination of the TA motif on one side of the transposon significantly reduces transposition while loss of TAs on both flanks of the transposon abolishes transposition.

The sequence requirements for each transposon's activity are likely to be unique to each species of transposon, but are essential for efficient DNA transposition.

In general, the 3′-terminal repeat will be the direct reverse complement of the 5′-terminal repeat. In some embodiments, non-direct (i.e. not 100% reverse complement identity, e.g. at least 90% or at least 95% reverse complement identity) may be tolerated, depending on the transposase used. In all cases, however, the relationship between the sequences of the inverted terminal repeats must be such that the transposon used is able to recognise both 5′- and 3′-ITRs, and able to excise the transposon.

The viral vector genome is one which is capable of being packaged into a virus capsid. Such encapsidation requires the presence of a viral packaging signal in the virus genome. In the viral vector of the invention, this viral packaging signal is present in the transposon, between the 5′- and 3′-inverted terminal repeats (ITRs).

The viral packaging signal is preferably obtained from or derived from the same virus from which the viral vector is obtained or is derived.

No packaging signals are present in the viral vector other than within the transposon.

The selective packaging of double-stranded DNA viruses such as adenovirus into virions late in infection involves the specific recognition of viral DNA sequences (i.e. packaging signals or packaging domains), and then condensation and encapsidation of the viral genome into pre-formed capsids. These packaging signals are therefore cis-acting elements which are required for the encapsidation of the viral genome.

For example, in the case of the standard adenoviral vector Ad5, specific packaging of viral DNA has been shown to be mediated by a packaging sequence which is located at the left end of the viral genome, i.e. between nucleotides 194 to 382 in Ad5 (Grable and Hearing, “Adenovirus type 5 packaging domain is composed of a repeated element that is functionally redundant”, J. Virol. 1990 May; 64(5): 2047-2056). This region contains at least five functionally-redundant domains, i.e. the “A repeats”, with AI, AII, AV, and AVI being the most important elements. Each of the A repeats fits a consensus motif and can function independently.

The Ad5 packaging signal is given herein as SEQ ID NO: 1. The Herpes Simplex Virus 1 packaging signal is given herein as SEQ ID NO: 2

The packaging signal and transgene (or transgene insertion site) are both flanked by the ITRs. The packaging signal may be placed 5′ of the transgene (or transgene insertion site) or 3′ of the transgene (or transgene insertion site).

Preferably, if the viral vector is an adenovirus viral vector, the packaging signal is placed at the 5′-end of the transposon just inside of the left ITR to allow the packaging signal to sit approximately in its native position in the adenovirus genome.

In the context of the current invention, if the transposase is able to remove the transposon from the virus genome during production of viral particles, the viral genome cannot be packaged because the transposed sequence contains the packaging signal which is essential for virus packaging. Therefore, only viral genomes which include the transposon can be packaged.

The viral vector of the invention comprises a transposon which comprises a transgene or a site for insertion of a transgene. The transgene may be any DNA sequence which is desired to be integrated into the genome of a mammalian cell. The DNA sequence may be a coding or non-coding sequence. It may be genomic DNA or cDNA. Preferably, the DNA sequence encodes a polypeptide, more preferably a therapeutic polypeptide. Preferably, the transgene is operably associated with one or more transcriptional and/or translational control elements (e.g. an enhancer, promoter, terminator sequence, etc.).

Examples of preferred therapeutic polypeptides include antibodies, CAR-T molecules, scFV, BiTEs, DARPins and T-cell receptors. In some embodiments, the therapeutic polypeptide is a G-protein coupled receptor (GPCR), e.g. DRD1. In some embodiments, the therapeutic polypeptide is an immunotherapy target, e.g. CD19, CD40 or CD38. In some embodiments, the therapeutic polypeptide is a functioning copy of a gene involved in human vision or retinal function, e.g. RPE65 or REP. In some embodiments, the therapeutic polypeptide is a functioning copy of a gene involved in human blood production or is a blood component, e.g. Factor IX, or those involved in beta and alpha thalassemia or sickle cell anaemia. In some embodiments, the therapeutic polypeptide is a functioning copy of a gene involved in immune function such as that in severe combined immune-deficiency (SCID) or Adenosine deaminase deficiency (ADA-SCID).

In some embodiments, the therapeutic polypeptide is a protein which increases/decreases proliferation of cells, e.g. a growth factor receptor. In some embodiments, the therapeutic polypeptide is an ion channel polypeptide.

In some preferred embodiments, the therapeutic polypeptide is an immune checkpoint molecule. Preferably, the immune checkpoint molecule is a member of the tumour necrosis factor (TNF) receptor superfamily (e.g. CD27, CD40, OX40, GITR or CD137) or a member of the B7-CD28 superfamily (e.g. CD28, CTLA4 or ICOS). Preferably, the immune checkpoint molecule is PD1, PDL1, CTLA4, Lag1 or GITR.

In some embodiments, the transposon does not comprise a transgene; it comprises a site for insertion of a transgene. Preferably, such an insertion site includes one or more restriction enzyme sites, e.g. a multiple-cloning site, more preferably 1, 2, 3, 4 or 5 restriction enzyme sites.

Adenovirus vectors can be generated by insertion or deletion of DNA in three regions of the adenovirus genome, namely in the E1 gene, in the E3 gene or in a short segment between the E4 gene and the end of the viral genome.

First generation adenoviral vectors were developed by replacing the gene sequence at the E1 region with a transgene. Deletion of the E1 gene inhibits viral replication inside host cells.

However, the transcription of the remaining viral genes resulted in early innate host cytokine transcription and adverse immune reactions. Thus, expression of the transgene was transient due to the immunogenic reaction that resulted in the destruction of all transduced cells.

Second and third generation adenovirus vectors were developed by deleting E1, E2 and E4 genes. The deletion of the afore-mentioned genes results in a reduced immune response against the vectors. Moreover, these vectors are also less immunogenic and show long-term gene expression within the host. As mentioned earlier, the E3 gene can also be deleted in order to accommodate large transgenes.

In the context of the current invention, the transposon is preferably inserted into an adenoviral vector in the E1 region where the E1A and E1B coding sequences are normally located.

The viral vector also comprises a nucleic acid (preferably DNA) encoding a transposase. The transposase is one which is capable of recognising the ITRs of the transposon.

In general, there will be a relationship between the transposase and the transposon: the transposase will generally be the transposase enzyme which is encoded by the wild-type transposon (or a derivative thereof). Hence the transposase will be one which recognises the ITRs of the transposon (i.e. is capable of binding to them) and, under appropriate conditions, is capable of excising the transposon from the viral vector and integrating it into the genome of a mammalian cell.

Preferably, the nucleic acid encoding the transposase is operably associated with one or more transcriptional and/or translational control elements, more preferably with an inducible or suppressible promoter element.

The expression of the transposase is preferably controlled by suitable means to prevent its undesired or untimely expression, or to suppress its expression level. In particular, it is desirable to prevent the expression of the transposase during the production of the viral vector (in order to prevent excision of the transposon.)

Preferably, the expression of the transposase is inducible or is repressed by the inclusion of an inducible or repressible regulatory (e.g. promoter) element.

The transposase transcription will be driven by a promoter, preferably a promoter with high expression levels in mammalian cells, e.g. the CMV, RSV, SV40, EF1 alpha, ubiquitin, HSV TK or PGK promoter.

Preferably, the promoter driving the transcription of the transposase is less than 500 base pairs in length, more preferably less than 250 base pairs. In some particularly preferred embodiments, the promoter comprises the minimal CMV promoter region.

Transcription of the nucleic acid encoding the transposase will be terminated by a poly-adenylation signal. Preferably, this signal is one which is highly active in mammalian cells, e.g. the bovine growth hormone poly adenylation signal or that from the SV40 virus genome.

In some embodiments, the transposase is placed under the control of an inducible promoter or inducible element. For example, the promoter may be one which is inducible with IPTG or lactose.

In some embodiments, the transposase promoter additionally comprises an inducible promoter element. Preferably, the inducible promoter element comprises a DNA sequence capable of binding proteins that can form a basal transcription complex and initiate transcription and a plurality of Tet operator sequences to which the Tet repressor protein (TetR) is capable of binding. In this bound state, tight suppression of transcription is obtained. However, in the presence of doxycycline, suppression is alleviated, thus allowing the promoter to gain full transcriptional activity. Such an inducible promoter element is preferably placed downstream of another promoter, e.g. the CMV promoter.

In some embodiments, the expression of the transposase is controlled at the translational level. For example, the nucleic acid encoding the transposase may incorporate a sequence which is complementary to a regulatory RNA, e.g. to a microRNA, siRNA or shRNA.

The nucleic acid encoding the transposase is preferably inserted into the viral vector adjacent to the ITRs, and more preferably 3′ to (i.e. downstream of) the most 3′ ITR sequence.

In an adenoviral vector, this more preferred position would be upstream from the E2B region.

The nucleic acid encoding the transposase is not placed within the region which is flanked by the ITRs.

The viral vector of the invention may comprise one or more viral genes (in addition to the viral packaging signal).

Preferably, the viral vector of the invention comprises at least the viral genes which must be present in the viral genome for the viral genome to be capable of being replicated and packaged, when other viral genes are provided in trans by packaging cell lines. In other words, the viral vector comprises all of the viral genes which cannot be provided in trans for the viral genome to be capable of being replicated and packaged.

The sequential process of gene transcription from the adenovirus genome reflects the protein requirements of the virus at each stage of the replication process. The transcription from the adenovirus genome is therefore divided into early and late events dependent on the timing of initiation of transcription from each viral promoter.

The first proteins produced from the virus genome are the E1A proteins. The E1A transcription unit produces multiple mRNA molecules through alternative splicing which in turn produce multiple proteins ranging from 6-36 kDa. The E1A proteins have two major roles within an infected cell. Firstly, they induce the cell to enter the S phase of the cell cycle to allow the efficient replication of the viral genome. Secondly, they induce transcription of the other early promoters within the viral genome through transactivation. These promoters control the production of the E1 B, E2, E3, E4 and E5 proteins. E1A expression is immediately followed by VA RNA and E1 B and E3 protein production. These proteins and RNA molecules help to prevent the development of an anti-viral response. These early events in viral replication help to shape the intracellular environment to allow the replication of the viral genome before packaging.

Later transcription events involve the production of structural proteins and proteins essential for cell lysis which are derived primarily from a single promoter (the major late promoter) which transcribes the late regions L1-L5. Cell lysis is dependent on the E3-11.6K protein (also termed the adenovirus death protein) which despite its labelling as an early gene is only produced late in infection and from the major late promoter.

The genome of the viral vector is one which is capable of being packaged. It is not necessary, however, for the viral vector genome to encode all of the polypeptides which are required for packaging; some of these polypeptides may be provided in trans by packaging cell lines.

In some embodiments, the viral vector comprises all viral genes which are necessary for replication of the viral genome.

In some embodiments, the viral vector comprises all viral genes which are necessary for packaging of the viral genome.

Preferably, the genome of the viral vector includes the following transcription units: E1A, E1B, E1B, IX, IVa2, E2B, L1, E2B, L2, L3, E2A-L, L4, U, L5 and E4.

In some embodiments, the viral vector does not comprise one or more of the early (E) proteins which are required by the wild-type virus for packaging during the early phase. For example, the viral vector (e.g. an adenoviral vector, e.g. Ad5) might not comprise the early proteins E1A and E1 B; these proteins would therefore need to be provided in trans by a packaging cell line during production of the viral vector.

The viral vector may additionally comprise one or more other elements, selected from the group consisting of genes conferring drug or antibiotic resistance, promoters, matrix attachment regions (MARs) or ubiquitous chromatin opening elements (UCOEs), restriction enzyme sites and core insulators.

In order to allow mammalian cells that contain the transposed DNA to be selected, the viral vector may comprise one or more genes conferring drug or antibiotic resistance.

In some embodiments, the viral vector additionally comprises a drug selection marker. Examples of such drug selection markers include genes encoding resistance to puromycin, blasticidin S, hygromycin, geneticin/G418 or zeocin.

In some embodiments, the viral vector additionally comprises one or more multiple-restriction enzyme sites. These sites will most preferably be Type II and either 6 or 8 base pairs in length.

The incorporation of a transgene into a host genome does not guarantee stable expression of the transgene per se, even though transposon insertions are biased towards actively transcribed loci. In some embodiments, therefore, it is advantageous to include an additional means of epigenetic stabilization for the transgene expression using core insulators. These core insulators, such as the HS4 core sequence derived from the chicken β-globin gene cluster, are able to resist heterochromatin spread and subsequent silencing of an adjoining DNA region.

Preferably, therefore, one or two core insulators are present in the transposon flanking the transgene and/or the virus packaging signal sequence. Preferably, the core insulators are HS4 cores.

DNA sequences can be subjected to epigenetic silencing in cells which prevents the expression of a therapeutic transgene. DNA that is attached to the nuclear matrix or scaffold is less prone to silencing.

Therefore, a viral vector of the invention may contain a matrix attachment region (MAR) and/or a ubiquitous chromatin opening element (UCOE).

The invention also provides a composition comprising a virus particle comprising a viral vector of the invention, together with one or more physiologically-acceptable carriers, excipients or diluents. Examples of suitable physiologically-acceptable carriers, excipients or diluents for use with virus particles are well known in the art.

Preferably, the composition comprises a virus particle comprising a viral vector of the invention in an aqueous buffer solution. Preferably, the aqueous buffer solution comprises MgCl₂ and/or glycerol.

The invention also provides a kit comprising a viral vector of the invention, wherein the kit additionally comprises one or more additional components selected from the group consisting of a virus packaging cell line that allows for the viral vector to be grown and packaged, and one or more DNA plasmids for aiding in the construction of the viral vector.

Preferably, the kit will comprise a plasmid containing the viral vector that contains sites for insertion of a transgene. More preferably there will be two plasmids: the viral vector plasmid and a shuttle plasmid to allow the easy manipulation of the viral vector. The shuttle plasmid will contain either regions of homology to the viral vector to allow homologous recombination to create a final viral vector, or restriction sites that are compatible with the viral vector to allow shuttling of DNA from the shuttle plasmid to the viral vector plasmid.

The kit may also contain materials for the purification of the viral particles such as those involved in the density banding and purification of viral particles, e.g. one or more of centrifuge tubes, benzonase, dialysis buffers and dialysis cassettes.

The invention also provides a mammalian cell comprising a viral vector of the invention.

The cells may be isolated cells, e.g. they are not present in a living animal. Examples of mammalian cells include those from any organ or tissue from humans, mice, rats, hamsters, monkeys, rabbits, donkeys, horses, sheep, cows and apes. Preferably, the cells are human cells. The cells may be primary or immortalised cells. Preferred human cells include HEK293, HEK293T, HEK293A, PerC6, 911, HeLa and COS cells. Most preferably, the human cells are HEK293, HEK293T, HEK293A, PerC6 or 911. Other preferred cells include CHO and VERO cells.

In a further embodiment, the invention provides a process for producing a modified mammalian cell, the process comprising the step:
(a) infecting a mammalian cell with a viral vector of the invention, whereby the mammalian cell then comprises the viral vector.

In a further embodiment, the invention provides a process for producing a mammalian cell with a modified genome, the process comprising the steps:
(a) infecting a mammalian cell with a viral vector of the invention, wherein the transposon comprises a transgene; and
(b) inducing expression of the transposase or removing repression of the transposase expression in the mammalian cell,
   whereby the transposase excises the transposon from the viral vector and integrates the transposon into the genome of the mammalian cell.

The processes of the invention are preferably carried out in vitro or ex vivo. Adenoviral vectors may be produced by several methods, the most common of which involves homologous recombination of adenovirus plasmids in either mammalian cells or microorganisms, including bacteria and yeast. Two plasmids, termed a shuttle plasmid and an adenoviral (also called backbone) plasmid, are recombined into a DNA molecule that incorporates sequences from both plasmids. This DNA molecule can then be transfected into mammalian packaging cell lines to generate adenovirus particles.

Herpes vectors may be produced by transfection of cells with DNA containing the packaging signal and origin of replication of Herpes Simplex Virus 1 and simultaneous infection with a replication defective virus that is able to provide all of the virus genes required for virus replication and packaging of Herpes Simplex Virus 1 DNA amplicons.

Transposon-mediated genomic integration may be assessed by colony formation assays and/or by sequencing of the host cell genome (or parts thereof). More generally however, the viral vectors used in embodiments of the invention will be non-integrating and cannot replicate. Therefore, if cells divide the DNA of the viral vector will be lost from one of the daughter cells. Therefore, over time most cells will not contain the viral vector. However, if DNA has been transposed from the viral vector into the host cell chromosome then dividing cells will replicate this DNA, whilst the un-transposed DNA of the viral vector will be lost. As such, the detection of cells that contain copies of the transposed DNA at a level higher than that of the copies of the viral vector DNA will indicate that DNA has been transposed to the host cell chromosome. This can be achieved by quantitative polymerase chain reaction (QPCR) analysis.

Integration of the transposon would be expected at canonical (such as TTAA sites for piggyBac-based viral vectors).

The insertion site for a transposon DNA can be found using a range of methods (e.g. Hui et al., Cell Mol Life Sci. 1998 December; 54(12):1403-11). Such techniques include inverse polymerase chain reaction (IPCR), novel Alu-PCR and vectorette- or splinkerette-PCR. These strategies allow for the cloning of flanking DNA regions of the integrants. Targeted gene-walking PCR, restriction-site PCR, capture PCR, panhandle PCR and boomerang DNA amplification are also possible methods that could be used. Inverse and splinkerette PCR are preferable methods of detection. GFP may be used as a reporter gene to quantify gene transfer efficiencies.

In a preferred embodiment, a viral vector is designed that comprises transcription units that allow the expression of two reporter genes that emit fluorescence at two different wavelengths when excited. One of these transcription units (e.g. encoding for EGFP) would be between the ITR sequences to be transposed and the other (e.g. encoding for RFP) would be outside of the ITRs but still within the viral vector at another location. One would expect that when this viral vector is used to infect target cells they would initially show the fluorescence of both reporter gene colours (e.g. yellow if expressing both green and red fluorescence). However, because only the reporter gene that is between the transposon ITRs will be transposed into the host cell chromosome, as the cells divide, the second reporter gene that is within the viral vector and cannot be transposed will be lost from the cells. As such the cells would eventually all become one colour (e.g. green). This could be measured by flow cytometry.

The disclosure of each reference set forth herein is specifically incorporated herein by reference in its entirety.

### EXAMPLES

The present invention is further illustrated by the following Examples, in which parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Example 1: Production of Adenoviral/piggyBac Vector

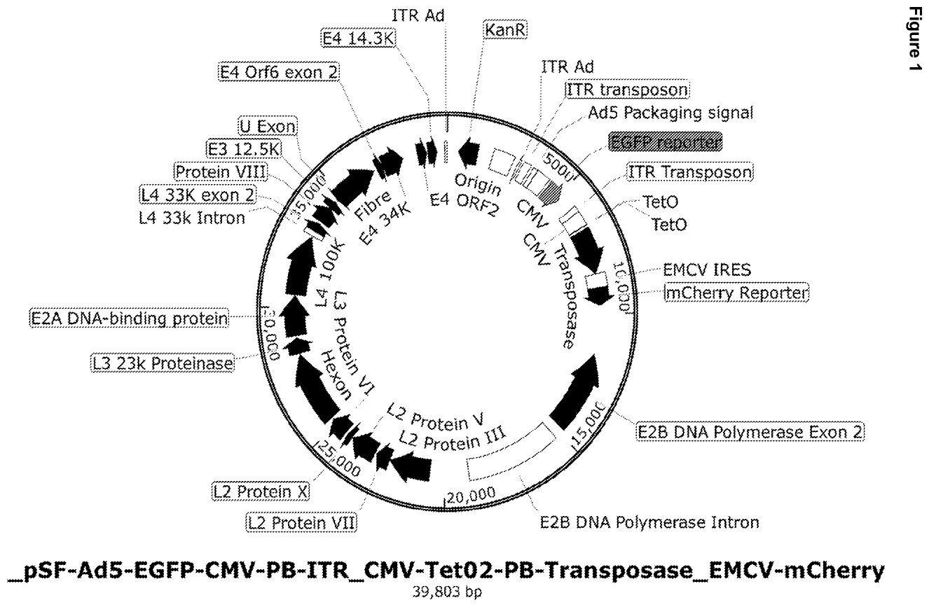

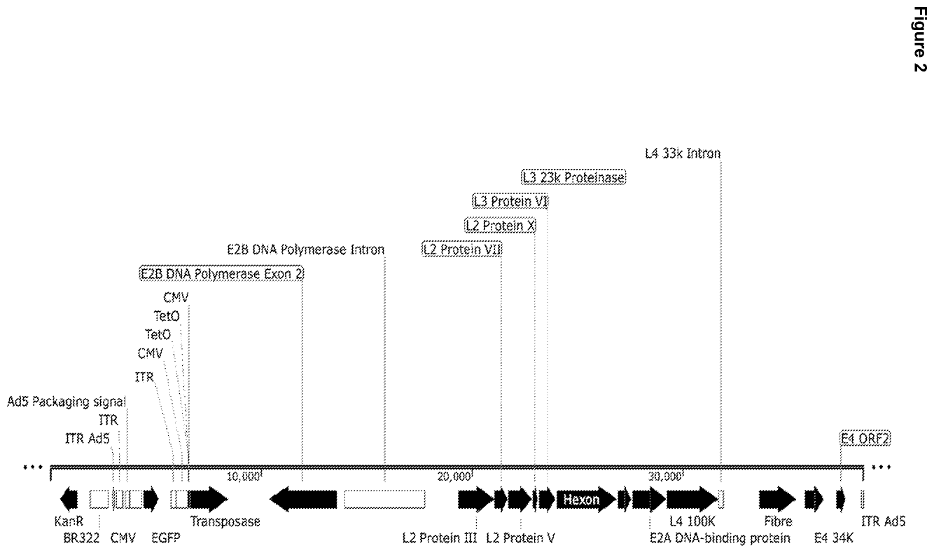

A shuttle vector was designed to contain (5′ to 3′) the following elements: the left ITR of the piggyBac transposon, the adenovirus packaging signal, a DNA promoter driving the expression of EGFP, a poly-adenylation signal, the right ITR from the piggyBac transposon, a second DNA promoter that can be bound by a tetracycline repressor, the coding sequence for the piggyBac transposase and a second poly-adenylation signal. This region of DNA was cloned into a suitable shuttle plasmid vector that contained regions of homology with an adenoviral plasmid vector. After restriction digestion of the adenoviral plasmid and the shuttle vector, the DNA molecules were joined by homologous recombination in bacteria to produce the viral vectors (see FIGS. 1-2).

The methods required for cloning and production of an adenoviral vector were as follows.

### Plasmid Amplification in Prokaryotic Systems

Transformation of Escherichia coli (E. coli) allows amplification of plasmids and subsequent screening for desired final DNA clones. DNA transformation was performed by thawing E. coli cells on ice, followed by the addition of 5-100 ng of DNA per transformation. Cells were incubated in ice for 20 minutes followed by 3 minutes heat-shock at 37° C. LB medium (0.9 mL per sample) was added to each tube and incubated for a further 15 minutes at 37° C. Samples were either poured into nutrient agar plates containing selection antibiotic or 100 μL of each sample was streaked out to allow individual colony isolation.

Successfully transformed clones were amplified and selected using antibiotic resistance using the appropriate antibiotic in growth media. Bacterial culture broth and plates containing either Kanamycin (50 μg/mL, Kanamycin Sulfate, Invitrogen, UK) or Ampicillin (50 μg/mL, Ampicillin Sodium salt, Sigma Aldrich) were prepared using Lennox LB Broth Base (20 g/L) and Lennox LB Agar (32 g/L) (Invitrogen, UK), respectively. Single bacterial colonies obtained on culture plates after transformation were picked and grown overnight in broth (5 mL mini-prep or 250 mL maxi-prep) at 37° C. with agitation (200 rpm) using a Sanyo orbital incubator (SANYO Electric Biomedical Co. Ltd, USA).

Plasmids were purified by mini-prep using the QIAprep Spin Miniprep Kit (Qiagen, UK) or maxi-prep using the Qiagen High Speed Maxi-prep kit (Qiagen, UK). Plasmid clones were selected and confirmed by diagnostic restriction digestion and DNA sequencing.

### Bacterial Cells

For the majority of small (<10 Kb) DNA constructs, DH5α E. coli bacteria (Invitrogen) were used to amplify plasmids. For larger (>10 Kb) constructs, XL10 Gold Ultracompetent E. coli bacteria (Strategene) were used to amplify DNA. These cells have been engineered in order to allow the efficient delivery of large DNA plasmids via heat-shock treatment and reduce bias towards selecting for small plasmids libraries. For growing DNA which was free of DAM and DMC methylation at key restriction sites, JM110 E. coli (Strategene) were used. These cells are deficient in methyl transferase enzymes and allow high purity methyl deficient DNA to be amplified. For recombination of DNA molecules, BJ5183 E. coli cells were used. These cells allow the recombination between two DNA molecules due to both the mutation of Endonuclease I and the expression of a RecET homologue which allows the efficient repair of double-strand breaks at homologous regions. All strains were grown in Lennox LB media or on LB agar plates at 37° C.

### DNA Restriction Digests (Preparative and Qualitative)

Restriction endonucleases were used to generate diagnostic digestion patterns or to produce DNA molecules with sticky (non-blunt) ends or blunt ends, allowing subsequent ligation of DNA fragments with other DNA molecules with either blunt or compatible overhangs. All digestions were performed according to conditions specified by the manufacturer (enzymes were purchased from New England Biolabs, UK or Promega, UK). Where digestions were performed for molecular engineering purposes, DNA fragments of correct sizes were visualised by gel electrophoresis and purified either from excised gel bands using the QIAEX II Gel Extraction Kit for large fragments (>8 Kb) (Qiagen, UK) and the MinElute Gel Extraction Kit for small fragments (Qiagen, UK), or, where it was unnecessary to resolve fragments of different sizes, purified directly from the digestion reaction (QIAquick PCR Purification Kit). HyperLadder™ I (Bioline Ltd, UK) was used as a molecular weight marker for fragments ranging from 1-10 Kb. For larger DNA fragments a Lambda Hind3 digest ladder was used (New England Biolabs, UK). For fragments less than 1 Kb a 100 base pair ladder was used (New England Biolabs, UK).

### DNA Ligation

Donor (insert) and recipient (vector) DNA were digested with single or double (for directional cloning) restriction enzyme(s) generating compatible overhangs for complementary base-pairing. Digested insert was gel-purified or cleaned up, while the vector was de-phosphorylated by treatment with 2 μL of calf intestinal alkaline phosphatase (37° C., 1 h) (Invitrogen, UK) to reduce background contamination resulting from vector re-ligation. Vectors were then purified using the MinElute Gel Extraction Kit (Qiagen, UK) or the QIAquick PCR Purification Kit (Qiagen, UK) using the manufacturer's protocols. All purified DNA was eluted in nuclease-free water. Ligation reactions were prepared using T4 DNA Ligase (New England Biolabs, UK) according to the manufacturer's protocol.

### Heat Shock Competent Cells

Heat Shock DNA competent E. coli cells were produced by inoculating a single colony of E. coli into 5 mL 2×YT broth media (16 g/L Bacto Tryptone, 10 g/L Yeast Extract, 5 g/L NaCl, pH 7.0 with 5M NaOH) which was shaken at 37° C. for 4 hours. This was transferred to 200 mL pre-warmed 2×YT media. When the culture reached OD 480 the cells were pelleted at 3000 rpm in a swing out centrifuge at 4° C. for 10 minutes. Cells were re-suspended in a total of 80 mL cold TFBI1 (30 mM KC₂H₃O₂, 100 mM RbCl, 10 mM CaCl₂.2H₂O, 50 mM MnCl₂.4H₂O, 15% v/v glycerol, adjusted to pH 5.8 using 0.2 M CH₃COOH, filter sterile). Cells were spun as above and re-suspended in 8 mL of TFBI2 (10 mM MOPS, 10 mM RbCl, 75 mM CaCl₂-2H₂O, 15% v/v glycerol, adjusted to pH 6.6 using 5 M KOH, filter sterile). 100 μl of cells were pipette into pre-chilled 1.5 mL microfuge tubes and frozen on dry ice. Tubes were then transferred to −80° C. for long term storage.

### Polymerase Chain Reaction (Cloning)

Polymerase chain reactions (PCR) were performed using oligonucleotide primers designed according to the requirements for molecular manipulation, sequencing or screening procedures (purchased from Sigma-Genosys, UK). Primer sequences employed for vector engineering and molecular characterisation purposes are described below.

For sub-cloning of DNA fragments generated by PCR amplification from DNA templates, high fidelity AccuPrime™ Pfx proofreading DNA polymerase (Invitrogen, UK) was used. For routine screening for the presence of specific DNA sequences by PCR, PCR SuperMix (Invitrogen, UK) was used. Primers were dissolved in de-ionised, nuclease-free water at 10 μM; 0.8-1 μL of each of the forward and reverse primers were used per PCR reaction. Reactions were carried out according to recommended protocols provided by the suppliers of enzymes and reagents. Where necessary, primer concentrations were varied and a gradient of annealing temperatures was run to determine optimal reaction conditions. PCRs were performed using a PTC-225 Peltier Thermal Cycler DNA Engine Tetrad (MJ Research, USA).

### Agarose Gel Electrophoresis

Size fractionation of DNA on 1% (w/v) agarose (Invitrogen, UK) gels containing ethidium bromide (Sigma-Aldrich, UK) (0.5 μg/mL) in 0.5×TAE buffer (4.84 g Tris-base, 1.09 g glacial acetic acid, 0.29 g EDTA in 1 L) enabled visualisation of DNA and size estimation, by relative comparison to commercially available molecular weight markers described above. DNA molecules from purified plasmids or fragments from PCR reactions or restriction digestions were subjected to gel electrophoresis (10 volts per cm gel length, 30-120 minutes) in horizontal DNA electrophoresis gel tanks (Bio-Rad Laboratories Ltd, UK). DNA bands were visualised using a gel and fluorescent imaging system (Alphalmager, Alpha Innotech Corporation, USA) under ultraviolet light.

### Double Stranded Deoxyribonucleic Acid Quantitation

DNA was quantified using a Nanodrop (Thermo Scientific, UK) spectrophotometer. Prior to analysis equipment was blanked using distilled water and then background readings were performed using the solution in which the DNA was dissolved (TE, EB or H₂O). Samples were tested in triplicate and data was interpreted using the ND-1000v 3.1.0 software according to the manufacturer's instructions. The final quantity of DNA was expressed as nanograms/microlitre.

### Calcium Phosphate Transfection for Adenovirus Recovery

SwaI-linearised adenoviral plasmids containing either recombinant adenoviral genomic DNA or the wild-type adenovirus genome are transfected into A549 lung carcinoma cells using calcium phosphate transfection. A549 cells are seeded in wells of a 6-well plate 24 h prior to transfection so that they are 70-80% confluent at the time of transfection. Calcium chloride (0.5 M, 250 μL) is mixed with DNA solution (20 μg in 250 μL sterile water) before being added drop-wise to 500 μL of 2×HEPES buffered saline (HBS) (Sigma, UK) while vortexing the tube containing HBS. The resulting transfection mixture (sufficient for the transfection of four 6-well plates) is allowed to stand for 1 minute before it is added drop-wise onto A549 monolayers which were growing in DMEM with 10% FBS (PAA Laboratories, Yeovil, UK) including penicillin (25 U/mL) and streptomycin (10 μg/mL) (Sigma Aldrich, UK).

The culture media containing the calcium phosphate transfection complex is removed at 24 h post-transfection, and transfected A549 cells are washed gently with PBS and collected by gentle pipetting; at this stage the transfected cells are not highly adherent and are amenable to removal without trypsinisation. Fresh DMEM media containing 2% FCS is added to each well seven days after plating; cytopathic effects (CPE) are observed in wells containing successfully transfected cells between 12 and 15 days post-transfection. Virus stocks are serially 10-fold diluted into 96 well plates. Single clones are picked and amplified in 10 cm dishes from which infectious supernatants are collected and stored as seed stock for further amplification and virus production.

### Virus Production and Purification by Banding on Caesium Chloride Gradients

Single virus clones are amplified in HEK-293 cells cultured in DMEM media containing 5% FCS, using approximately 15-25 confluent 175 cm² monolayers for each purification. HEK-293 monolayers containing virus-packed cells are harvested (72 hours post-infection, when CPE is observable but infected cells are not lysed) by gentle agitation and pelleted cells are re-suspended in infectious supernatant (16 mL, volume accommodated in three banding columns), then lysed by three freeze-thaw cycles to release virus particles. The mixture containing lysed cells and free virus particles is centrifuged at 245 g (10 min, 4° C.). The mixture is incubated on ice for 60 min, followed by centrifugation at 800 g (10 min, 4° C.). The pellet is discarded and the supernatant (containing virus particles) is loaded onto centrifuge tubes (Ultra-clear™ Beckman Centrifuge tubes, Beckman Coulter UK Ltd) containing a caesium chloride (CsCl) gradient. The gradients are centrifuged (25,000 rpm, 10° C., 120 min without deceleration using a Beckman L8-70M Ultracentrifuge with rotor type SW40 TI) (Beckman Coulter, Inc, USA). Two discrete bands are obtained after centrifugation: a faint band higher in the column containing ‘empty’ viral particles, and a thicker, opaque lower band containing intact infectious viral particles. The virus is harvested by puncturing the tube below the level of the virus band with an 18-gauge needle and extracting the desired band into a syringe.

CsCl is subsequently removed by consecutive dialyses in buffers (500 mL, 4° C.) containing 50 mM HEPES, 1×PBS, 0.1 g/L CaCl₂, 0.2 g/L (initial dialysis) or 0.1 g/L (final dialysis) MgCl₂ and 10% glycerol at pH 7.8, using a 3-15 mL dialysis cassette for the initial dialysis (Pierce Slide-A-Lyzer® Dialysis Cassette, Pierce Biotechnology, Inc., USA) after which the virus is recovered and treated with Benzonase DNA nuclease (6 μL/mL, Novagen, UK) for 30 minutes at room temperature. The virus is subsequently re-banded by CsCl gradient centrifugation and dialysed overnight in the final dialysis buffer using a 0.5-3 mL cassette (Pierce Slide-A-Lyzer® Dialysis Cassette, Pierce Biotechnology, Inc., USA). For each dialysis, the buffer is changed and replaced with fresh buffer after 1 hour and 2 hours, followed by an overnight dialysis. Virus obtained after the final dialysis is aliquoted and stored immediately at −80° C.

### Double-Stranded DNA Measurement for Adenovirus Quantitation

Adenoviral DNA concentrations are determined using the PicoGreen assay (Quant-iT™ PicoGreen® dsDNA Reagent, Molecular Probes, Invitrogen). The assay contains a fluorescent nucleic acid probe for double-stranded DNA (dsDNA), allowing quantification of adenoviral genome content. Appropriate dilutions of virus stock solutions (10 to 100-fold) are made and are transferred (15 μL) to a solution containing 1×TE buffer (255 μL) and 0.5% sodium dodecyl sulphate (SDS) in 1×TE (30 μL). Samples are incubated at 56° C. (30 min) to disrupt virus particles. Six four-fold serial dilutions of known concentrations of the bacteriophage lambda DNA provided in the Quant-iT™ PicoGreen® Kit are carried out (highest concentration at 1 μg/mL), allowing the construction of a standard curve (last standard blank) from which the DNA content in unknown samples are calculated. The PicoGreen reagent is diluted 200-fold in 1×TE buffer. 100 μL of the diluted reagent is placed in wells of a black 96-well plate (Corning, UK) for each standard or unknown sample. 50 μL of appropriately diluted standards and samples are added to the wells in duplicate. The plate is read in a Wallac 1420 Victor² multi-label counter, using the ‘Fluorescein 485/535 nm, 1 second’ program for analysis. The number of viral particles present in each sample is calculated on the basis that 1 μg of DNA is approximately equal to 2.7×10¹⁰ adenoviral particles.

### Calculation of Plaque Forming Units of Adenoviral Preparations Using the Tissue Culture Infectious Dose 50 (TCID50) Method

The tissue culture infectious dose 50 (TCID50) method (Karber 1931) is used to estimate the number of infectious virus particles, or plaque forming units (pfu), and is based on the development of cytopathic effects (CPE) in HEK-293 cells after infection with 10-fold serially diluted samples of virus preparation. This method is described in full in the manual for the AdEasy non-replicating virus platform available from QBiogene (France).

### Adenovirus Preparations

All adenoviruses are grown in HEK-293 cells, purified by double banding in CsCl gradients as described above. Viral particle (vp) number is determined by measuring DNA content using a modified version of the PicoGreen assay (Invitrogen, Paisley, UK) (Mittereder et al. 1996). Infectivity is calculated using the TCID₅₀ system with the KARBER statistical method (Karber 1931) and is used to estimate the adenovirus titer (TCID₅₀ units/mL) and corrected to determine plaque forming units/mL (pfu/mL). Expected adenovirus preparation characteristics are as follows: Ad5 WT: 1.13×10¹² vp/mL, 1.98×10¹¹ pfu/mL and particle:infectivity (P:I) ratio of 5.6; Ad5-mir122: 1.29×10¹² vp/mL, 2.01×10¹¹ pfu/mL and particle:infectivity (P:I) ratio of 6.4.

### Maintenance of Cell Lines

HEK293 human embryonic kidney cells are obtained from the European Collection of Cell Cultures (Porton Down, UK), and maintained in DMEM media with 10% FCS (PAA Laboratories, Yeovil, UK) including penicillin (25 U/mL) and streptomycin (10 mg/mL) at 37° C. in 5% CO₂ in a humidified incubator.

### Real Time (Quantitative) PCR (Q-PCR) for Ad5

The Q-PCR methodology for measurement of adenoviral particles has been previously described (Green et al. 2004). Briefly, viral DNA from infected cells or tissue samples is extracted using a mammalian genomic Genelute DNA extraction kit (Sigma). Reactions are performed using Applied Biosystems master mix following the manufacturer's protocol. The cycles are as follows: 94° C. 10 min then 40 times at 94° C. 30 s, 60° C. 1 min. Primers sequences for targeting Ad5 fiber are: Forward primer—5′ TGG CTG TTA AAG GCA GTT TGG 3′ (SEQ ID NO: 4) (Ad5 32350-32370 nucleotides) and reverse primer—5′ GCA CTC CAT TTT CGT CAA ATC TT 3′ (SEQ ID NO: 5) (Ad5 32433-32411 nt) and the TaqMan probe—5′ TCC AAT ATC TGG AAC AGT TCA AAG TGC TCA TCT 3′ (SEQ ID NO: 6) (Ad5 32372-32404 nt), dual labelled at the 5′ end with 6-carboxyfluorescein and the 3′ end with 6-carboxytetramethylrhodamine. The results are analyzed with the Sequence Detection System software (Applied Biosystems). Standard curves for tissues and cells are prepared by spiking samples of cell lysate or tissue homogenate with serial dilutions of known concentrations of virus particles followed by extraction and analysis of each sample separately by Q-PCR as described above.

### Example 2: Testing for Integration of Transposon

A viral vector is designed that contains transcription units that allow the expression of two reporter genes that emit fluorescence at two different wavelengths when excited. One of these transcription units encodes EGFP; this lies between the ITR sequences to be transposed. The other encodes RFP; this lies outside of the ITRs but still within the viral vector at another location. This viral vector is used to infect target cells. The cells initially show the fluorescence of both reporter gene colours, i.e. yellow when expressing both green and red fluorescence. However, because only the reporter gene that is between the transposon ITRs is transposed into the host cell chromosome, as the cells divide, the second reporter gene (RFP) that is within the viral vector and cannot be transposed will be lost from the cells. As such, the cells eventually all become one colour (i.e. green). This is measured by flow cytometry as follows:

Target HEK-293 suspension cells are infected at a multiplicity of infection of 1 with the adenoviral vector encoding the transcription cassettes for EGFP and MCherry (RFP), where the EGFP cassette is between two ITRs to be transposed into the target cells. 12 hours post-infection, a portion of cells representing less than 10% of the total population is analysed for GFP and RFP expression using a BD Accuri flow cytometer using the FL1 and FL3 channels with cell activation using two lasers with laser excitation at 488 nm and 640 nm. Green and red fluorescence profiles are detected. Each day, a portion of the cell population is read in the same way until 10 days post-infection. The EGFP signal is maintained within the cell population due to integration, whilst the MCherry signal reduces over time as the viral vector backbone is lost from the cells.

### REFERENCES

Edgar, R., M. Domrachev, et al. (2002). “Gene Expression Omnibus: NCBI gene expression and hybridization array data repository.” Nucleic Acids Research 30(1): 207-210.
Green, N. K., C. W. Herbert, et al. (2004). “Extended plasma circulation time and decreased toxicity of polymer-coated adenovirus.” Gene Therapy 11(16): 1256-1263.
Karber, G. (1931). “50% end-point calculation.” Arch Exp Pathol Pharmak 162: 480-483.
Mittereder, N., K. L. March, et al. (1996). “Evaluation of the concentration and bioactivity of adenovirus vectors for gene therapy.” Journal of Virology 70(11): 7498-7509.
Pfaffl, M. W. (2001). “A new mathematical model for relative quantification in real-time RT-PCR.” Nucleic Acids Research 29(9).
Vanrooijen, N. and A. Sanders (1994). “Liposome-Mediated Depletion of Macrophages—Mechanism of Action, Preparation of Liposomes and Applications.” Journal of Immunological Methods 174(1-2): 83-93.

ADDITIONAL SEQUENCES
Adenovirus Packaging Signal sequence
SEQ ID NO: 1
TAGTGTGGCGGAAGTGTGATGTTGCAAGTGTGGCGGAACACATGTAAG
CGACGGATGTGGCAAAAGTGACGTTTTTGGTGTGCGCCGGTGTACACA
GGAAGTGACAATTTTCGCGCGGTTTTAGGCGGATGTTGTAGTAAATTT
GGGCGTAACCGAGTAAGATTTGGCCATTTTCGCGGGAAAACTGAATAA
GAGGAAGTGAAATCTGAATAATTTTGTGTTACTCATAGCGCGTAATAT
TTGTCTAGGGCCGCGGGGACTTTGACCGTTTACGTGGAGACTCGCCCA
GGTGTTTTTCTCAGGTGTTTTCCGCGTTCCGGGTCAAAGTTGGCGTTT
TATTATTATAGTCAGCTGACG
Herpes Simplex Virus 1 packaging signal
SEQ ID NO: 2
TCCCGCGGCCCCGCCCCCCACGCCCGCCGCCGCGCGCGCGCACGCCGC
CCGGACCGCCGCCCGCCTTTTTTGCGCGCGCGCGCGCCCGCGGGGGGC
CCGGGCTGCCCGCCGCCGCCGCTTTAAAGGGCCGCGCGCGACCCCCGG
GGGGTGTGTTTTGGGGGGGGCCCGTTTTCGGGGTCTGGCCGCTCCTCC
CCCCGG
piggBac transposase
SEQ ID NO: 3
MGSSLDDEHILSALLQSDDELVGEDSDSEISDHVSEDDVQSDTEEAFI
DEVHEVQPTSSGSEILDEQNVIEQPGSSLASNKILTLPQRTIRGKNKH
CWSTSKSTRRSRVSALNIVRSQRGPTRMCRNIYDPLLCFKLFFTDEII
SEIVKWTNAEISLKRRESMTGATFRDTNEDEIYAFFGILVMTAVRKDN
HMSTDDLFDRSLSMVYVSVMSRDRFDFLIRCLRMDDKSIRPTLRENDV
FTPVRKIWDLFIHQCIQNYTPGAHLTIDEQLLGFRGRCPFRMYIPNKP
SKYGIKILMMCDSGTKYMINGMPYLGRGTQTNGVPLGEYYVKELSKPV
HGSCRNITCDNWFTSIPLAKNLLQEPYKLTIVGTVRSNKREIPEVLKN
SRSRPVGTSMFCFDGPLTLVSYKPKPAKMVYLLSSCDEDASINESTGK
PQMVMYYNQTKGGVDTLDQMCSVMTCSRKTNRWPMALLYGMINIACIN
SFIIYSHNVSSKGEKVQSRKKFMRNLYMSLTSSFMRKRLEAPTLKRYL
RDNISNILPNEVPGTSDDSTEEPVTKKRTYCTYCPSKIRRKANASCKK
CKKVICREHNIDMCQSCF

1. A viral vector comprising:
(a) a transposon comprising inverted terminal repeats (ITRs) at its 5′- and 3′-ends, the ITRs flanking
(i) a packaging signal for the viral vector genome, and
(ii) a transgene or a site for insertion of a transgene;
and
(b) a nucleic acid encoding a transposase, wherein the transposase is one which is capable of recognising the ITRs of the transposon,
wherein the nucleic acid encoding the transposase is not flanked by the transposon ITRs.

2. The viral vector as claimed in claim 1, wherein the nucleic acid encoding the transposase is operably associated with one or more transcriptional and/or translational control elements.

3. The viral vector as claimed in claim 1, wherein the nucleic acid encoding the transposase is operably associated with an inducible or suppressible promoter element.

4. The viral vector as claimed in claim 1, wherein the viral vector is an adenoviral vector or an Ad5 vector.

5. The viral vector as claimed in claim 4, wherein the viral vector is replication-defective or replication-incompetent.

6. The viral vector as claimed in claim 5, wherein the viral vector has one or more of the early genes E1, E2 and E3 deleted.

7. The viral vector as claimed in claim 1, wherein the transposon is one which is capable of integrating into a vertebrate genome or into a mammalian genome.

8. The viral vector as claimed in claim 7, wherein the transposon is obtained or derived from a Class II DNA, Subclass I transposon.

9. The viral vector as claimed in claim 7, wherein the transposon is obtained or derived from piggyBac, Sleeping Beauty or Tol2.

10. The viral vector as claimed in claim 1, wherein the packaging signal is placed at the 5′-end of the transposon just inside of the left ITR.

11. The viral vector as claimed in claim 1, wherein a transgene is present within the viral vector, preferably wherein the transgene is a therapeutic polypeptide.

12. The viral vector as claimed in claim 1, wherein the viral vector comprises all of the viral genes which cannot be provided in trans for the viral genome to be replicated and packaged.

13. The viral vector as claimed in claim 1, wherein the viral vector comprises all of the viral genes which are necessary for replication of the viral genome and/or all viral genes which are necessary for packaging of the viral genome.

14. The viral vector as claimed in claim 1, wherein the viral vector additionally comprises one or more elements selected from the group consisting of genes conferring drug or antibiotic resistance, restriction enzyme sites, core insulators, matrix attachment regions (MARs) and ubiquitous chromatin opening elements (UCOEs).

15. The composition comprising a virus particle comprising a viral vector as claimed in claim 1, together with one or more carriers, excipients or diluents.

16. The kit comprising a viral vector as claimed in claim 1, wherein the kit additionally comprises one or more additional components selected from the group consisting of a virus packaging cell line that allows for the viral vector to be replicated and packaged, and/or one or more DNA plasmids for aiding in the construction of the viral vector.

17. The mammalian cell comprising a viral vector as claimed in claim 1.

18. A process for producing a modified mammalian cell, the process comprising the step:
(a) infecting a mammalian cell with a viral vector as claimed in claim 1,
whereby the mammalian cell then comprises the viral vector.

19. The process for producing a mammalian cell with a modified genome, the process comprising the steps:
(a) infecting a mammalian cell with a viral vector as claimed in claim 1, wherein the transposon comprises a transgene; and
(b) inducing expression of the transposase or removing repression of the transposase in the mammalian cell,
whereby the transposase excises the transposon from the viral vector and integrates the transposon into the genome of the mammalian cell.
